(19) United States
(12) Patent Application Publication
(10) Pub. No.: US 2023/0329816 A1
(43) Pub. Date: Oct. 19, 2023

(54) ENDOSCOPIC DEVICES, SYSTEMS AND METHODS

(71) Applicant: Taurean Surgical, Inc., Flower Mound, TX (US)

(72) Inventors: Pitale Ashok Rahul KUMAR, Telangana (IN); Sunil Varghese MANJILA, Ann Arbor, MI (US)

(21) Appl. No.: 18/300,080

(22) Filed: Apr. 13, 2023

Related U.S. Application Data
(60) Provisional application No. 63/330,615, filed on Apr. 13, 2022.

(51) Int. Cl. A61B 34/37; A61B 34/20; A61B 34/30

(52) U.S. Cl. CPC A61B 34/37; A61B 34/20; A61B 34/30; A61B 2034/2048; A61B 2034/301

(57) ABSTRACT

An example endoscope includes an elongated handle extending between first and second ends. An elongated insertion part is coupled to and extends from a proximal end portion thereof to terminate in a distal end portion of the insertion part. A housing is coupled to the first end of the handle, the housing including an actuator therein configured to cause movement of the distal end portion of the insertion part. A mount coupling can be on the housing or the handle. The mount coupling is adapted to connect to a mating part of a support structure, such that when connected to the mating part of the support structure, the endoscope is held in a fixed position with respect to the support structure, and when not connected to the mating part of the support structure, the endoscope can be manipulated manually independently from the support structure.

### RELATED APPLICATION

This application claims the benefit of priority to U.S. App. No. 63/330615, filed Apr. 13, 2022, and entitled ENDOSCOPIC DEVICES, SYSTEMS AND RELATED METHODS, which is incorporated herein by reference in its entirety.

### TECHNICAL FIELD

This disclosure relates to surgical devices and, more particularly, to endoscopic devices, systems and methods.

### BACKGROUND

An endoscope is an illuminated optical, typically slender and tubular instrument used to look deep into the body by way of openings such as the mouth, nose or anus, and used in procedures called an endoscopy. Endoscopes use tubes are configured to transfer illumination in one direction and high-resolution images in real time in the other direction, resulting in minimally invasive surgeries. In some examples, the placement and/or use of endoscopes within the patient can be robotically controlled or robotically assisted. Endoscopes can be used for performing interventions with respect to various parts of the body.

### SUMMARY

This disclosure relates to surgical devices and, more particularly, to endoscopic devices, systems and methods.

In one example, an endoscope includes an elongated handle extending between first and second ends. An elongated insertion part is coupled to and extends from a proximal end portion thereof to terminate in a distal end portion of the insertion part. A housing is coupled to the first end of the handle, the housing including an actuator therein configured to cause movement of the distal end portion of the insertion part. A mount coupling can be on the housing or the handle. The mount coupling is adapted to connect to a mating part of a support structure, such that when connected to the mating part of the support structure, the endoscope is held in a fixed position with respect to the support structure, and when not connected to the mating part of the support structure, the endoscope can be manipulated manually independently from the support structure.

In another example a method includes disconnecting an endoscope from an attachment to a robotic arm. The attachment to the robotic arm includes a mount coupling, which is on a housing or handle of the endoscope and releasably coupled to a mating part of the robotic arm to fix the endoscope with respect to the robotic arm. The endoscope includes an elongated insertion part having a distal end portion, and the endoscope includes an actuator configured to move the distal end portion of the elongated insertion part in response to a steering control signal. The method also includes manually manipulating the endoscope independently from the robotic arm. The method can also include re-connecting the endoscope to the robotic arm by connecting the mount coupling to the mating part of the robotic arm, such that when connected to the robotic arm, the endoscope is held in a fixed position with respect to the mating part of the robotic arm and moveable commensurate with movement of the robotic arm.

In yet another example, an endoscope includes an elongated handle extending between first and second ends. An elongated insertion part is coupled to and extends from a proximal end portion to terminate in a distal end portion. A housing is coupled to the first end of the handle. The housing includes an actuator therein configured to cause movement of the distal end portion of the insertion part, in which the distal end portion includes a continuum mechanism extending longitudinally between proximal and distal ends thereof. The continuum mechanism is configured to enable the distal end thereof to deflect over a generally spherical range of motion. The endoscope also includes a mount coupling on the housing or the handle. The mount coupling is adapted to connect to a mating part of a support structure, such that when connected to the support structure the endoscope is held in a fixed position with respect to the mating part of the support structure, and when not connected to the support structure the endoscope can be manipulated manually independently from the support structure. A steering interface device can be mounted on the handle, in which the steering interface device is configured to provide a steering signal based on an applied user input. An actuator controller is configured to control the actuator responsive to the steering signal to move the distal end portion in a respective direction relative to a central axis of the insertion part. The endoscope can also include one or more endoscopic tools at the distal end of the endoscope.

In a further example, an endoscopic system includes the endoscope described herein, and a robotic arm that constitutes the support structure having the mating part to which the mount coupling can be connected. Thus, the mating part of the robotic arm can be coupled to the robotic arm to hold the endoscope at a fixed position with respect to the robotic arm, in which the endoscope is configured to move commensurate with movement of the robotic arm. The mount coupling can also be removed from the mating part of the robotic arm to enable a user to move and use the endoscope independently from the robotic arm.

### BRIEF DESCRIPTION OF DRAWINGS

FIGS. 1, 2, 3, and 4 are different views showing an example of an endoscope.

FIG. 5 is a bottom perspective view showing controls for the endoscope.

FIGS. 6, 7, 8 and 9 show an example of the endoscope with the handle oriented at different positions.

FIGS. 10, 11, 12, and 13 illustrate a latch coupling in different positions for coupling the endoscope apparatus to a support structure, such as a robotic arm.

FIG. 14 depicts an example of the endoscope apparatus coupled to a robotic arm for robotic manipulation.

FIG. 15 depicts an example of the endoscope apparatus detached from the robotic arm for manipulation by a user.

FIGS. 16 and 17 depict a handle portion oriented at different angles with respect to an insertion portion of the endoscope.

FIG. 18 is a side view of a partial assembly view showing pull wires and drive mechanisms for steering a distal end of the endoscope.

FIGS. 19 and 20 are different enlarged views of the actuator assembly and pull wires shown in FIG. 18.

FIG. 21 is a partial assembly view of the endoscope showing a distal end portion having a continuum manipulator.

FIGS. 22 and 23 depict an example of the distal end portion having the continuum manipulator.

FIG. 24 depicts a front view of the continuum manipulator.

FIG. 25 is a perspective view showing an example of a distal end that can be implemented for the endoscope.

### DETAILED DESCRIPTION

The techniques of this disclosure generally relate to endoscopic devices, systems and methods.

As an example, an endoscope includes an elongated handle extending between first and second end portions. A housing is coupled to the first end portion of the handle, and an elongated insertion part extends from the housing to terminate in a distal end portion. One or more actuators can reside in the housing and be configured to cause deflection of the distal end portion of the insertion part in a desired direction. For example, an actuator controller is configured to control the actuator responsive to a steering signal to deflect the distal end portion in a respective direction.

The endoscope can include an arrangement of one or more user input devices (e.g., mounted on the handle) configured to control respective endoscopic functions, including the deflection of the distal end portion. For example, a steering interface (e.g., a joystick and/or an arrangement of push buttons) configured to provide a steering signal based on an applied user input. A trigger can also be provided on a side of the handle configured to provide an activation signal for implementing a respective endoscopic function.

In an example, the endoscope can be releasably attached to a support structure, such as a robotic arm implemented as part of a robotic endoscope system. The endoscope can include a mount coupling on the motor housing or the handle. The mount coupling is adapted to connect to a mating part of the robotic arm (e.g., an end effector), such that when connected to the support structure, the endoscope is held in a fixed position with respect to the robotic arm. Thus, the robotic arm can move in three-dimensional space responsive to robotic control signals, which provides corresponding movement of the endoscope. Also, when the endoscope is released from (e.g., not coupled to the robotic arm), the endoscope can be held by a user and manipulated manually independently from the support structure (e.g., separate independent use from the robotic arm).

To further facilitate manual use of the endoscope in an ergonomic manner, the endoscope can include an adjustment coupling (e.g., a spheroid joint linkage) between the housing and the handle. The adjustment coupling enables angular movement of the handle with respect to the housing and/or the insertion part, increasing the ergonomics of the endoscope.

In some examples, the distal end portion of the endoscope can include a longitudinally extending continuum mechanism. The continuum mechanism is configured to allow the distal end thereof to deflect and smoothly move over a generally spherical range of motion. The continuum mechanism can include a plurality of discs axially spaced along a longitudinal axis between respective proximal and distal ends thereof. In an example, the continuum mechanism includes a helical sidewall in which each turn of the helical sidewall defines a respective one of the discs. A tubular outer sheath of a flexible material can be disposed over an outer surface of the continuum mechanism.

In order to steer the endoscope, the endoscope can include an arrangement of one or more pull wires extending through the insertion part and being fixed at the distal end of the continuum mechanism. In an example, each pull wire extends through a respective set of axially aligned holes of the discs to hold the discs substantially orthogonally with respect to the longitudinal axis. The continuum mechanism deflects in a direction according to which of the pull wire or wires are actuated by the actuator. In an example, the actuator is implemented as including an arrangement of rotary motors configured to adjust the length of the respective pull wires and cause corresponding deflection of the continuum mechanism responsive to activation of the user input devices.

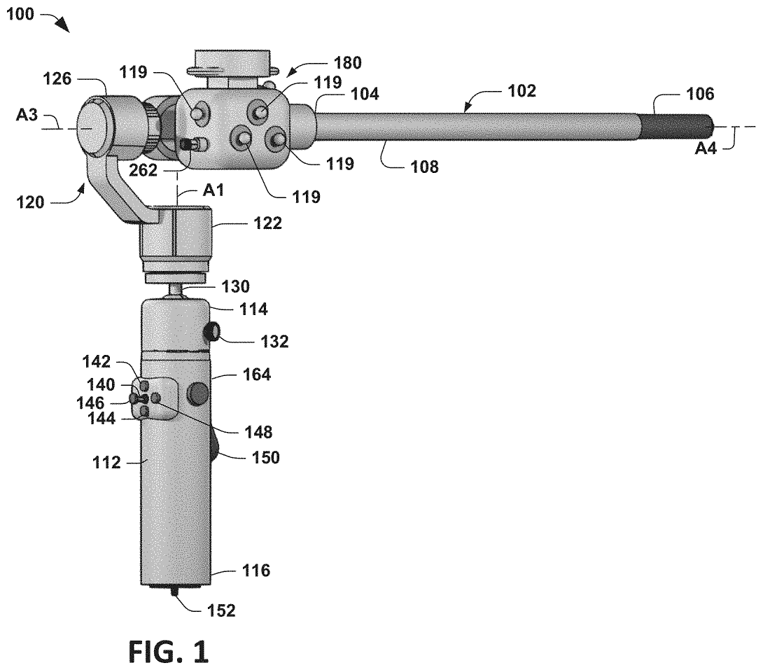

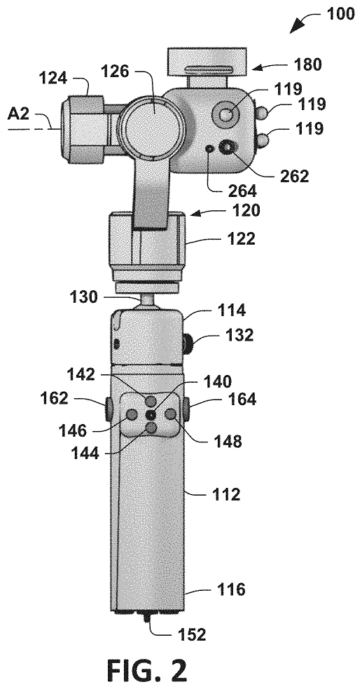

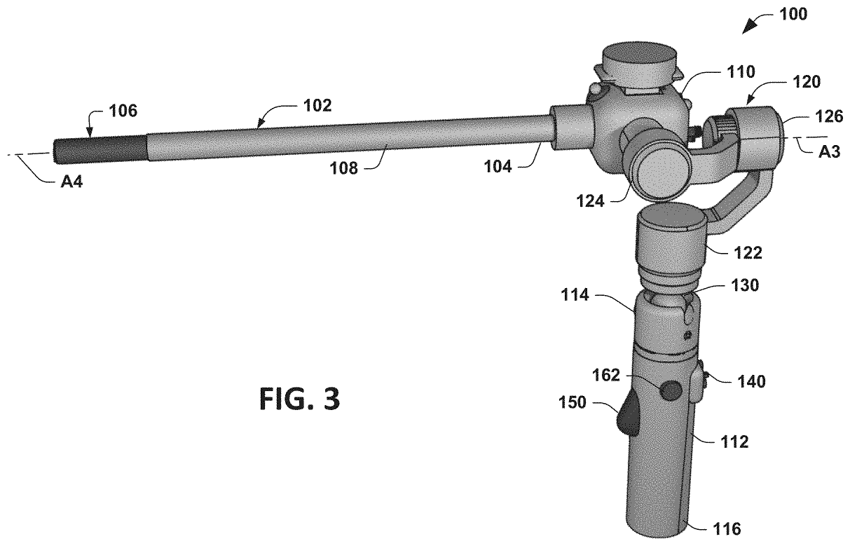

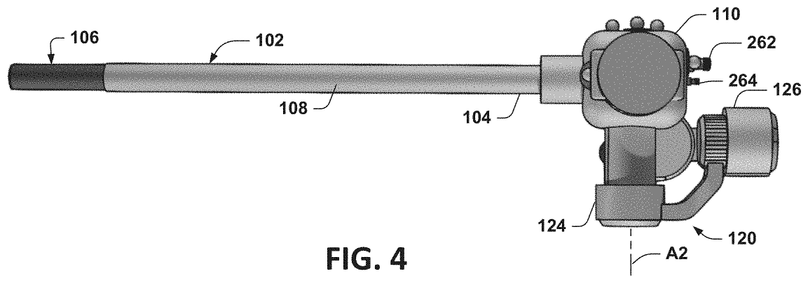

FIGS. 1, 2, 3, and 4 are different views showing an example of an endoscope 100. FIGS. 1 and 3 are opposing side elevation views of the endoscope 100. FIG. 2 shows a rear view and FIG. 4 is a top view of the endoscope. As described herein, the endoscope can be used as a standalone endoscope or implemented as part of a robotic endoscopic system. It will also be appreciated that the endoscope 100, endoscopic systems and methods disclosed herein are applicable not only to ear, nose and throat (ENT) surgery and neurosurgery but can also be used for surgeries performed in other parts of the body, such as abdominal, gynecological, urological, and the like.

The endoscope 100 includes an insertion part 102 having elongated body extending from a proximal end portion 104 and terminating in a distal end portion 106. For example, the insertion part 102 includes a proximal tubular body portion 108 having a sidewall formed of a rigid material, such as stainless steel or a polymer material. The tubular body portion 108 can have one or more lumens extending through the body portion to provide a passage for various tools, pull wires that may reside within the insertion part 102, such as disclosed herein. The tubular body portion 108 can have a circular or polygonal cross-sectional shape.

In an example, the tubular body portion 108 is implemented as a medical grade stainless steel hollow tube having an outer diameter ranging from about 4 mm to 10 mm. The body portion can include one or more channels or subchannels (e.g., lumens of flexible material) extending longitudinally through the body portion to receive electrical wires, cables, optical fibers and the like. Additionally or alternatively, one or more subchannels can be used to transport fluids (e.g., irrigation/cleaning fluids, suction, etc.) or enable passage of other tools (e.g., grippers, cutting tools, etc.) for use at or near the distal end of the endoscope

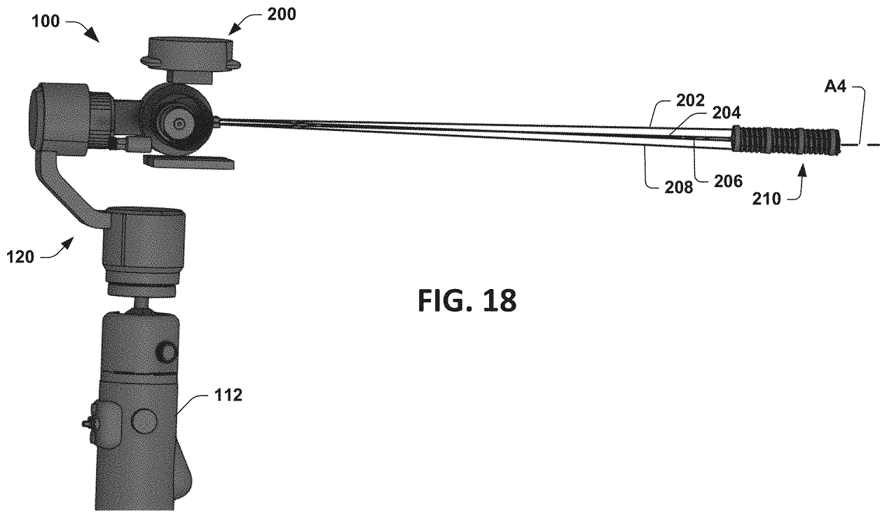

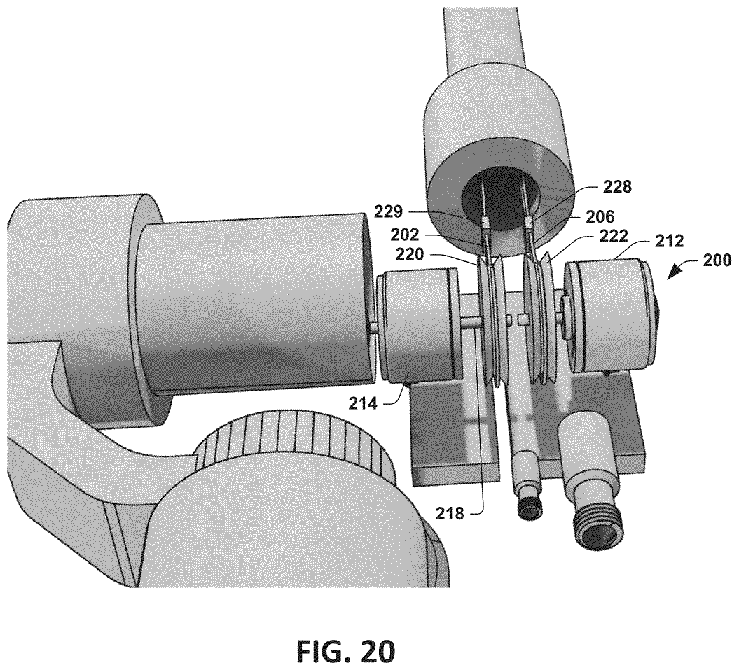

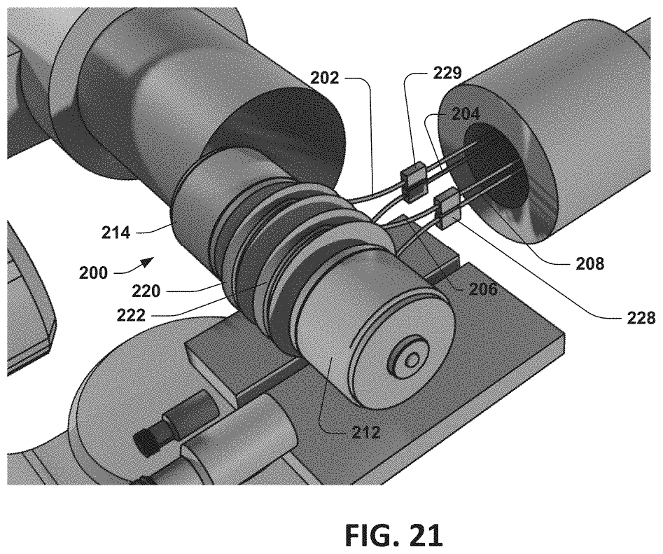

The proximal end 104 of the insertion part 102 can be coupled to a housing 110. The housing 110 is configured and arranged to enclose at least a portion of an actuator assembly. The actuator assembly includes one or more actuators (e.g., motors) configured to cause deflection of the distal end portion 106 of the insertion part 102, such as disclosed herein (see, e.g., FIGS. 18-21). The endoscope 100 also includes control electronics configured to control each actuator or other endoscopic functions in response to user inputs. The endoscope 100 also includes an elongated handle 112 extending between first and second end portions 114 and 116. While the example of FIGS. 1-4 shows the handle with a circular cross-sectional shape between its end portions 114 and 116. Other shapes can be used in other examples. Finger grooves or other ergonomic features also can be implemented on the handle 112.

As shown in FIGS. 1-4, the housing 110 and handle 112 can include an arrangement of optical markers 119. The markers 119 can be used for accurate estimation of object recognition, object positioning and orientation of the endoscope in 3D space. For example, the position and orientation of the endoscope in 3D space can be used derived from the marker and by an associated robotic endoscope system to move the endoscope and/or to implement endoscopic control functions in the 3D space.

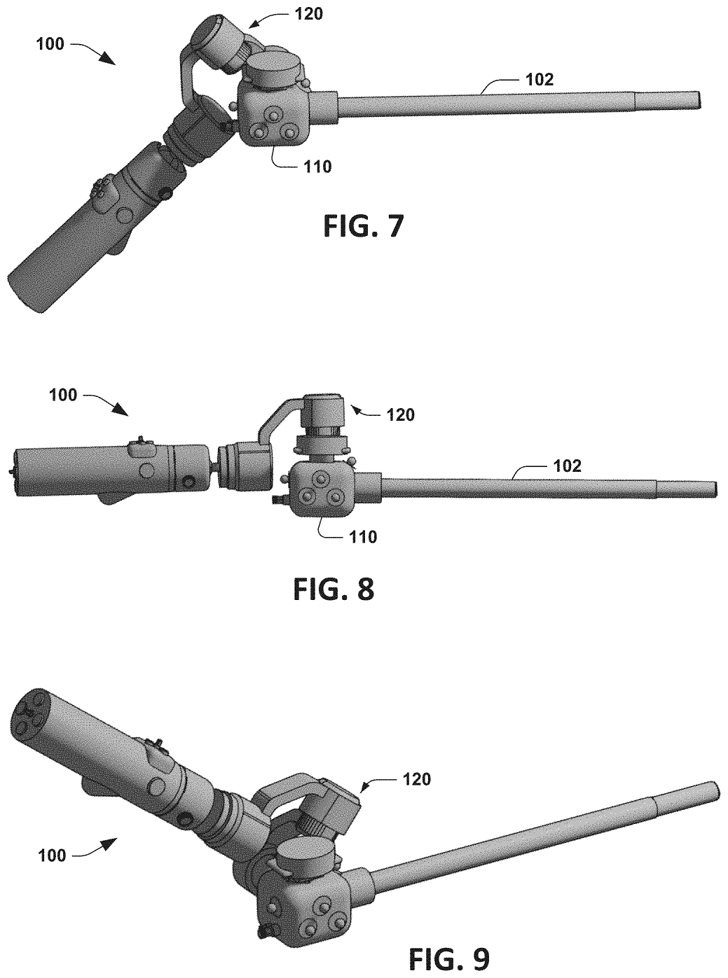

In some examples, the endoscope 100 includes a gimbal apparatus 120 configured to stabilize a position and orientation of the insertion part of the endoscope, such as during use. As shown in FIGS. 1-9, the gimbal apparatus 120 is coupled between the housing 110 and the end portion 114 of the handle 112. In other examples, the gimbal apparatus 120 may be modified in its arrangement, such as implemented within the housing 110, or it may be omitted altogether from the endoscope 100.

In the example shown in FIGS. 1-9, the gimbal apparatus 120 is a motorized gimbal apparatus having a plurality of motors 122, 124 and 126 as well as an arrangement of sensors and control electronics (not shown). Each of the motors 122, 124 and 126 is arranged and configured to rotate the housing 110 about a respective axis of rotation (shown as A1, A2 and A3), in which the respective axes of rotation are orthogonal to each other. In the example of FIGS. 1-4, A1 is coaxial with a longitudinal axis of the handle 112 and A3 is parallel (and can be coaxial) with a longitudinal axis of the insertion part 102. A1, A2 and A3 can be orthogonal with respect to each other in three-dimensional space and thus define a three-dimensional coordinate reference frame for the endoscope 100.

By way of example, the gimbal apparatus 120 also includes an inertial measurement unit (IMU), which can be located in or on the housing 110. The IMU measurement unit is configured to perform multi-axis inertial sensing and provide inertial sensor data representative of position, orientation and/or motion of the housing 110. Thus because the rigid proximal portion 108 of the insertion part 102 is fixed with respect to the housing 110, the inertial sensor data is also representative of position and/or motion of the insertion part 102. As an example, the IMU can include a gyroscope and acceleration sensor configured to measure angular rotation and acceleration, respectively, for each of the orthogonal axes A1, A2 and A3 and provide corresponding gyroscope data and acceleration data. In another example, the IMU includes a gyroscope, accelerometer, delta angle, delta velocity, and barometer sensor for each of the orthogonal axes A1, A2 and A3. An example of an IMU that can be implemented in the endoscope is the ADIS16480 iSensor® device available from Analog Devices. Other IMU devices can be used in other examples. The IMU can include one or more other sensors, such as configured to acquire position and orientation information for the endoscope 100.

The gimbal apparatus 120 can also include a gimbal controller (e.g., hardware and software located in the housing 110 and/or the handle 112) configured to provide motor control signals to control the plurality of motors 122, 124 and 126 for stabilizing the housing 110 and insertion part 102 responsive to the inertial sensor data. For example, the gimbal apparatus 120 can be used to hold the insertion part in a constant and steady position and orientation regardless of the position of the handle 112 with respect to the housing 110 and insertion part 102. FIGS. 6, 7, 8 and 9 show the handle at different angular positions relative to the housing 110 and insertion part 102. In this way, as a user moves the handle to a desired position for holding the endoscope, the gimbal apparatus 120 can stabilize the insertion part 102 at a substantially fixed spatial orientation.

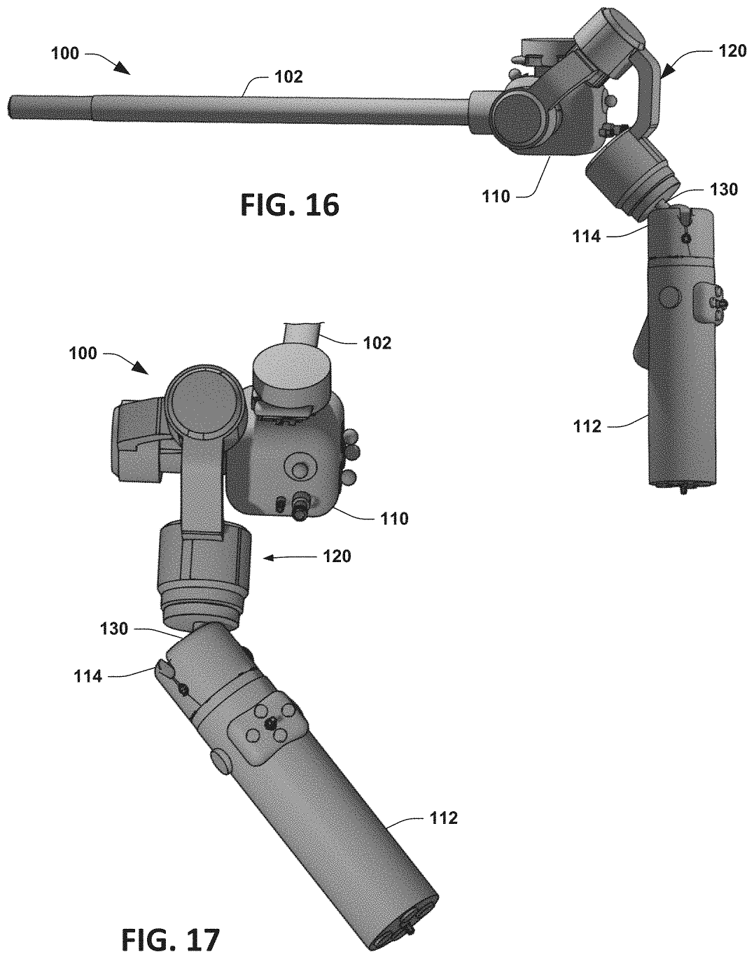

Referring back to FIGS. 1-4 as well as to FIGS. 16-17, in some examples, the endoscope 100 includes an adjustment coupling 130 between the handle 112 and the gimbal apparatus 120. In examples where the gimbal apparatus 120 is omitted from the endoscope 100, the adjustment coupling 130 can be coupled between the handle 112 and the housing 110. The adjustment coupling 130 is configured to enable a user to adjust the angular position of the handle 112 with respect to the housing 110 and/or the insertion part 102 (e.g., over about 270 degrees of rotation). The adjustment coupling 130 thus provides the user flexibility in using the device in any hand and wrist position with reduced strain. For example, the adjustment coupling 130 is implemented as a spheroid linkage joint (e.g., ball swivel joint), in which a spherical ball is mounted within a spherical receptacle formed in the distal end portion 114 of the handle. A shaft extends from the spherical ball and is coupled to an end of the gimbal apparatus. Alternatively the shaft and spherical ball can be integrally formed with the housing of the gimbal apparatus. A locking mechanism 132 can be configured to hold (at least temporarily fix) the relative position between the adjustment coupling 130 and the handle 112 and/or vary the stiffness between the handle 112 and the adjustment coupling 130. For example, the locking mechanism 132 is implemented as a fastener, such as threaded knob, T-bar lock, a bolt, a pin or another type of fastener adapted to secure (e.g., fix) the position of the adjustment coupling 130 with respect to the handle 112. Other types of adjustment couplings can also be used, such as hinge, pivot joint, articulating joint, a track or other coupling arranged and configured to enable a user to adjust the relative angular position of the handle 112 with respect to insertion part 102. The locking mechanism 132 can also be unlocked to release the adjustment coupling 130 with respect to the handle 112 so a user can move the housing 110 to a desired position and orientation relative to the handle 112.

As a further example of enhanced ergonomics, the endoscope 100 can include an arrangement of one or more user input devices configured to control respective endoscopic functions. In the example of FIGS. 1-5, the endoscope includes a plurality of user input devices to enable manual controls from the endoscope when it is being used manually (independently from a robotic arm) as well as during robot-assisted use when the endoscope is coupled to a robotic arm.

In the example of FIGS. 1-5, a first joystick 140 is located on a proximal side surface of the handle 112 at a location between respective end portions 114 and 116 (e.g., closer to the end portion 114). An arrangement of push buttons 142, 144, 146 and 148 surround the joystick 140. The joystick 140 and push buttons 142, 144, 146 and 148 can be configured to provide a steering signal based on an applied user input to control deflection of the distal end portion 106. For instance, the joystick 140 provides the steering signal to command continuous movement of the distal end portion and in a corresponding direction responsive to activation of the joystick away from its neutral starting position. Each of the push buttons 142, 144, 146 and 148 can provide a steering signal to command a predetermined amount of movement of the distal end portion in a corresponding direction responsive to activation (e.g., by pressing) a respective button. That is, pressing one of the buttons 142, 144, 146 and 148 provides a command signal to cause a preset amount of deflection in a corresponding direction (e.g., sub-millimeter motion, such as μm motion). The amount of deflection responsive to activation of any of the user input devices can be programmable in response to a user input and stored in memory of the endoscope to control the deflection accordingly.

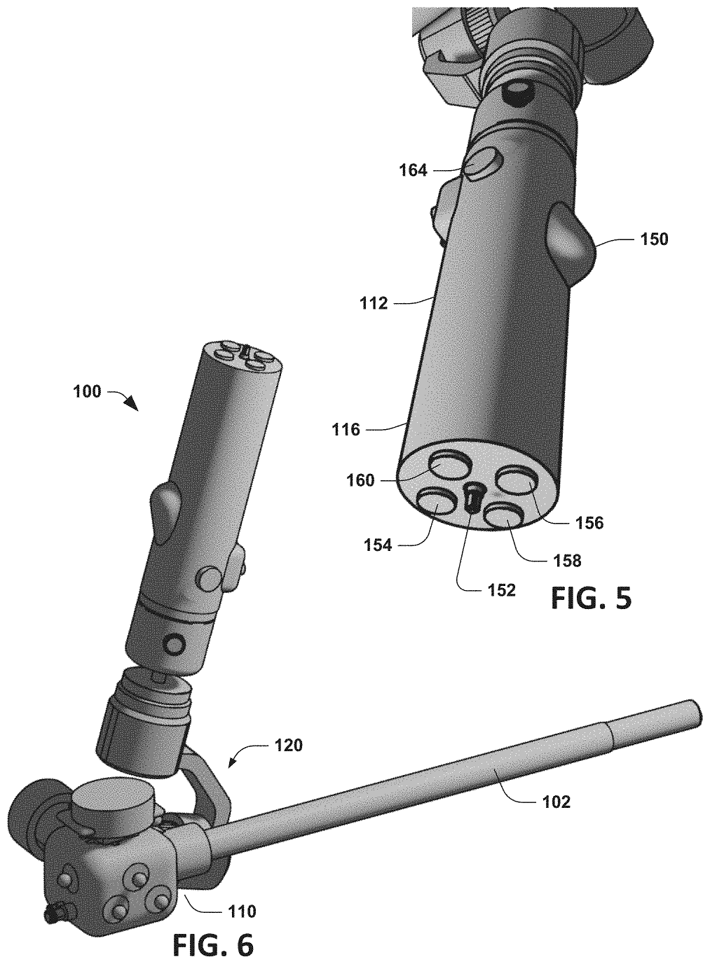

In an example, the joystick 140 and push buttons 142, 144, 146 and 148 can be arranged on a control pad that protrudes from an outer surface of the handle 112. A trigger 150 can also be provided on a side of the handle 112 opposite the control pad. The trigger 150 is configured to provide an activation signal for implementing a respective endoscopic function. In some examples, another set of actuator user input devices can be implemented on the end portion 116. For example, a second joystick 152 and push buttons 154, 156, 158 and 160 are arranged on the free end of the handle 112, such as shown in FIG. 5. The joystick 152 and push buttons 154, 156, 158 and 160 can be configured to perform the same functions as the joystick 140 and push buttons 142, 144, 146 and 148 on the side of the handle. The endoscope 100 can include an actuator controller (e.g., hardware and/or software residing in the housing 110 and/or handle 112) configured to control one or more actuators responsive to the steering signals (from user input devices 140, 142, 144, 146, 148, 152, 154, 156, 158 or 160) to deflect the distal end portion 106 in a corresponding direction relative to a central longitudinal axis A4 of the insertion part 102.

One or more additional side buttons 162 and 164 can be provided to control other endoscopic functions. Each of the endoscopic functions activated or controlled in by any the user input devices 140-164 can be predetermined (e.g., be preprogrammed) or be user-programmable in response to a user input instruction mapping selected functions to the respective devices. Tables 1 and 2 below describe a set of example endoscopic functions that can be mapped to the respective user input devices. Other functions can be implemented in other examples.

TABLE 1

| HANDLE SIDE | |
|---|---|
| BUTTONS | ENDOSCOPIC FUNCTION |
| Trigger 150 | Ejects water onto the lenses and clears the |
| | field by removing debris |
| Joystick 140 | Bends the tip of the endoscope in the direction |
| | where the joystick is pushed |
| Button 142 | Provides micro-millimeter bending of the |
| | endoscope tip upwards |
| Button 144 | Provides micro-millimeter bending of the |
| | endoscope tip downwards |
| Button 146 | Provides micro-millimeter bending of the |
| | endoscope tip to the left |
| Button 148 | Provides micro-millimeter bending of the |
| | endoscope tip to the right |
| Left big | Activating or deactivating the gimbal |
| button 162 | stabilization system |
| Right big | Imaging control functions, such as white |
| button 164 | balance, image correction and other imaging/ |
| | video parameters |

TABLE 2

| HANDLE BASE | |
|---|---|
| BUTTONS | ENDOSCOPIC FUNCTION |
| Joystick 152 | Bends the tip of the endoscope in the direction where |
| | the joystick is pushed |
| Buttons 154 | Provides micro-millimeter bending of the endoscope tip |
| | upwards |
| Button 156 | Provides micro-millimeter bending of the endoscope tip |
| | downwards |
| Button 158 | Provides micro-millimeter bending of the endoscope |
| | tip to the left |
| Button 160 | Provides micro-millimeter bending of the endoscope |
| | tip to the right |

As disclosed herein, the endoscope 100 is configured to enable hybrid use of the endoscope, including manual, robotic-assisted or robotic usage of the endoscope. For example, if a surgeon wants to use two hands for surgery, the surgeon can attach the endoscope 100 to a robotic arm, and if the surgeon wants to hold the endoscope during the surgery, then the surgeon simply has to detach the endo scope from the robotic arm and hold the handle 112 during the procedure.

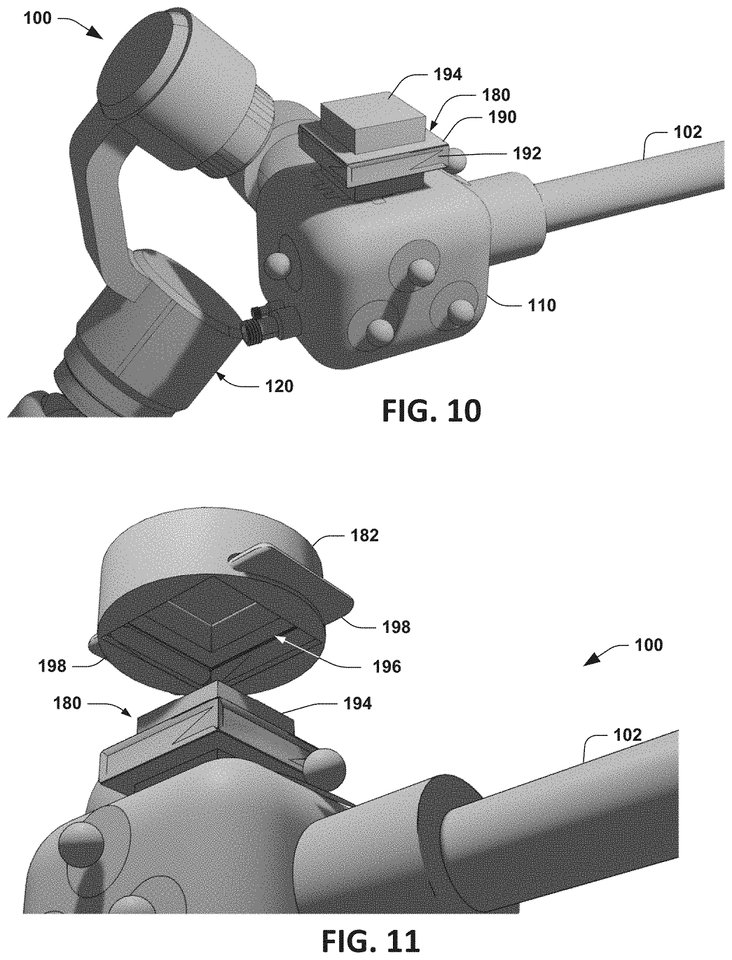

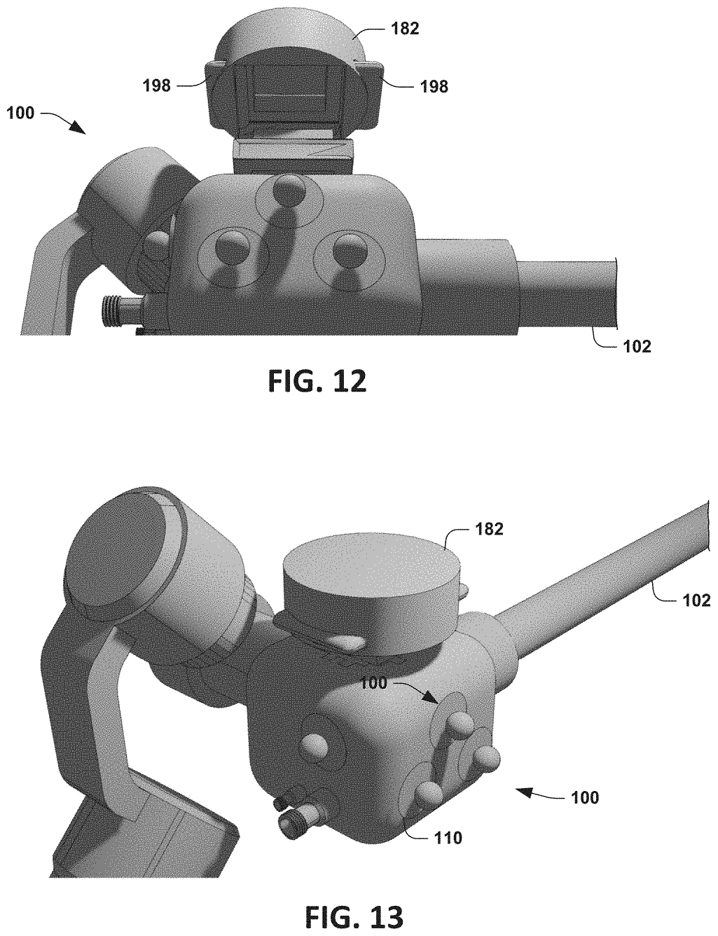

For example, with reference to FIGS. 10, 11, 12, 13, 14 and 15, the endoscope 100 includes a mount coupling 180 adapted to connect to a mating part 182 of a support structure, such as a robotic arm 184 of a robotic endoscope system 186. As shown in the example of FIGS. 10-15, the mount coupling 180 is implemented on a top side surface of the housing 110 from which the insertion part 102 extends. In other examples, the mount coupling can be provided on another surface of the housing or on other parts of the endoscope 100, such as the handle 112. The mating part 182 is attached to or formed on the support structure to which the endoscope is to be mounted.

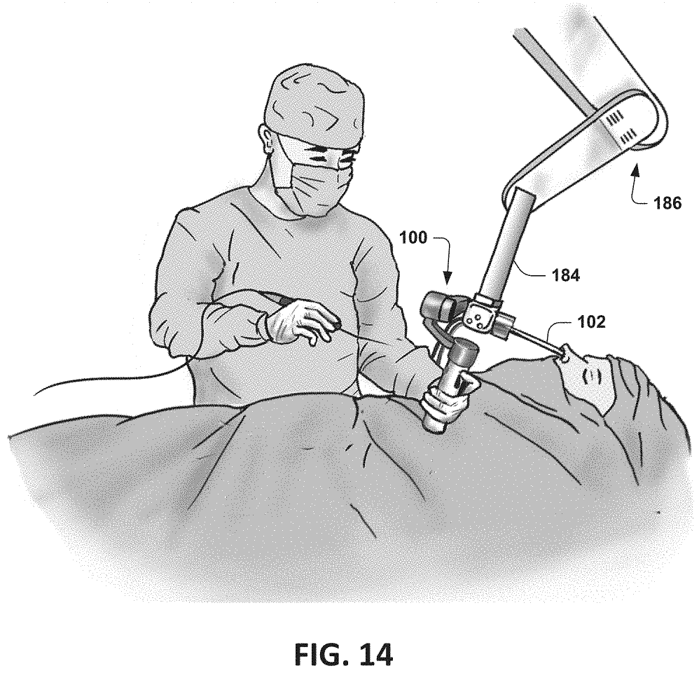

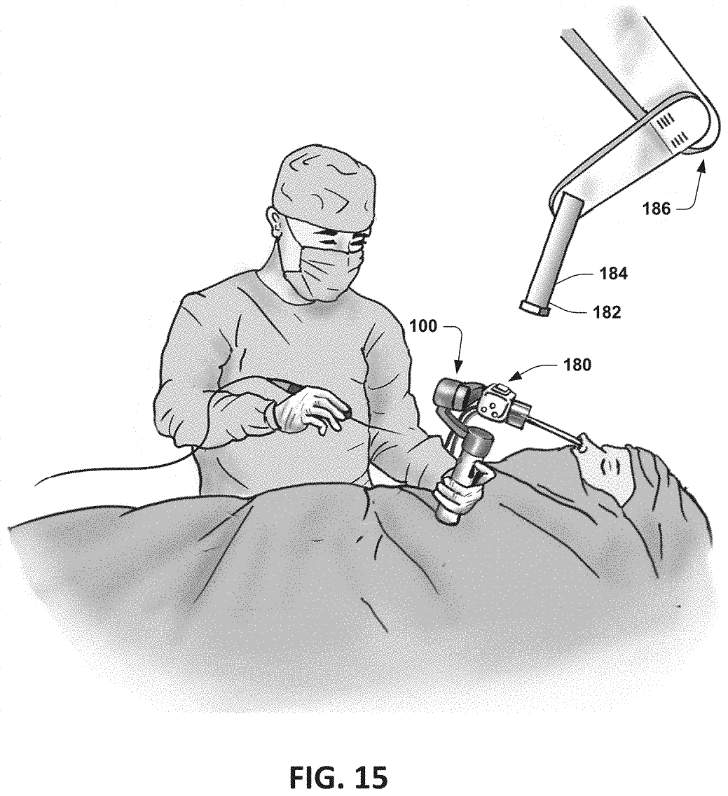

In the examples of FIG. 14, the mating part 182 is coupled to the end effector of the robotic arm 184. The mount coupling 180 and mating part 182 are configured to releasably attach the endoscope 100 to the support structure (e.g., the robotic arm 184) so that, when connected to the support structure (e.g., the robotic arm 184), the endoscope is held in a fixed position and orientation with respect to the support structure. In the example of FIG. 15, the endoscope 100 is shown detached from the support structure (e.g., the robotic arm 184). When detached, the endoscope 100 can be manipulated manually by the user independently from mechanical constraints of the support structure (e.g., the robotic arm 184). Regardless of whether the endoscope 200 is coupled to or detached from the robotic arm 184, the user can activate one or more buttons or other user interface elements implemented on the endoscope to perform respective endoscopic functions, such as disclosed herein.

In the example of FIGS. 10-13, the mount coupling 180 is a male part and the mating part 182 is a female part. The mount coupling 180 thus includes a connector part 190 having a size and configuration adapted to be received within a receptacle 196 of the mating part 182. The mount coupling 180 can also include a magnet 194, such as on a top side surface of the connector part 190, configured to magnetically attach to a corresponding magnet of opposite polarity within the mating part 182. In an example, the connector part 190 has a cuboidal shape with receiving features (e.g., receptacles) formed in one or more sidewalls 192. The receiving features are configured and arranged to receive retaining tabs of the mating part 182. For example, the retaining tabs and receiving features form a fastener adapted to releasably lock the mount coupling 180 with respect to the support structure 184 and to enable quick detachment of the endoscope from the support structure in response to release of the retaining tabs. The retaining tabs 198 can be inwardly mechanically biased (e.g., with one or more springs) to fit into the receiving features and anchor the endoscope onto the support structure 184. A radially outward force can then be applied to the retaining tabs for removing the endoscope from the robotic arm. In other examples, the male and female parts could be switched, or the releasable coupling between the endoscope 100 and the robotic arm 184 could be implemented in the absence of male and female mating parts.

One skilled in the art will understand appreciate various other types of mechanical couplings (e.g., Velcro, latches, straps, etc.) that can be used, additionally or alternatively, to releasably mechanically couple the endoscope 100 with respect to the robotic arm 184. The mount coupling thus provides the surgeon with the ability to quickly and easily remove the endoscope 100 from the robotic arm 184 and use it manually based on needs. Additionally, by providing such a coupling at the end effector of the robotic arm allows for different endoscope configurations or other types of devices to be coupled to the robotic arm and increase the versatility of the robotic system.

In an example, a delta robot can be provided at the interface where the mount coupling 180 attaches to the robotic arm. The delta robot is a three legged robot having fixed and moveable platforms. The “fixed platform” can be coupled to the robotic arm and the “moving platform” can include the mating part 182 to which the mount coupling 180 is attached (and can be removed from). The advantage of such a system is that it creates a movement similar to a human wrist with similar freedom.

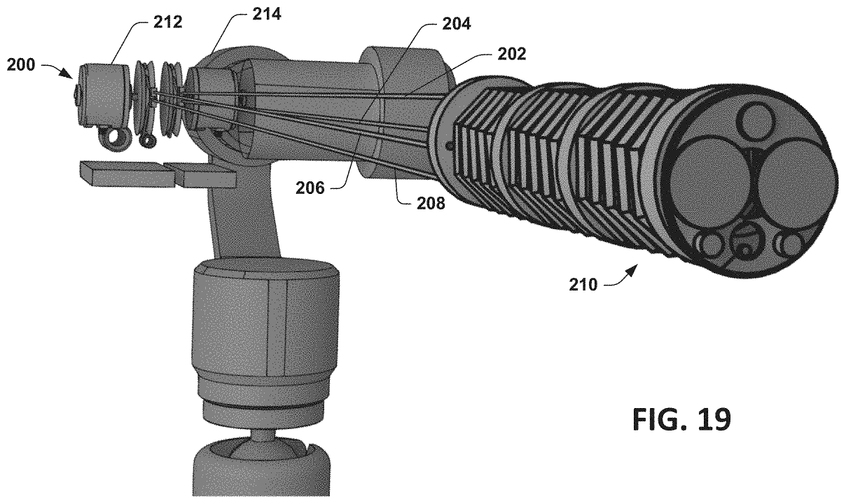

FIGS. 18 and 19 are side and front views of the endoscope 100 with certain parts (e.g., housing 110, tubular body portion 108 and distal outer sheath) removed to show the arrangement and configuration of interior components. In the example of FIGS. 18 and 19, the interior components shown includes an actuator assembly 200, an arrangement of pull wires 202, 204, 206 and 208, and a continuum mechanism 210. The pull wires 202, 204, 206 and 208 extend between the actuator assembly 200 and the continuum mechanism 210, and a distal end of each pull wire is anchored at a distal end of the continuum mechanism 210. The actuator assembly 200 includes one or more actuators configured to actuate the respective pull wires to cause the distal end portion 106 to deflect in a respective direction, such as disclosed herein.

For example, each actuator is implemented as a rotary actuator, such as a rotary motor configured to drive a pulley or other rotatable track based on a motor control signal. Examples of rotary actuators include stepper motors, brushless DC (BLDC) motors, permanent magnet synchronous motors, AC induction motors, brush-type DC motors. In another example, the actuator can be implemented as a linear actuator configured to provide motion in one degree of freedom for driving a respective pull wire. Examples of linear actuators include stepper-motor actuators, linear motor actuators and piezo-motor actuators. The type of actuator can be selected according to the type pull wire being used.

In the example of FIGS. 18-21, the actuator assembly 200 includes a rotary motor 212 and 214 for each pair of pull wires. For instance, pull wires 202 and 204 form one pair of pull wires and wires 206 and 208 form a second pair. The actuator assembly 200 also includes a drive pulley 220 and 222 coupled to a respective motor shaft 216 and 218. The proximal end portion of pull wire pair 202, 204 is on one drive pulley 220 and a proximal end portion of the other pull wire pair 206, 208 is on the other drive pulley 222. Each drive pulley 220, 222 is thus adapted to rotate about an axis based on rotation of the motor shaft by the motor to adjust the respective lengths of the pull wires 202, 204, 206 and 208 and cause a corresponding deflection of the distal end portion. Rotation of a respective drive pulley 220, 222 reduces a length of one pull wire and correspondingly increases a length of the other pull wire that is coupled to the respective drive pulley.

Each motor 212 and 214 has a respective motor shaft 216 and 218 and is configured to rotate responsive to a motor control signal. For example, an actuator controller is configured to provide the motor control signal responsive to a steering command signal, such as provided by one or more user input devices (e.g., user input devices 140, 142, 144, 146, 148, 152, 154, 156, 158 or 160). The actuator controller can also implement motor controls responsive to sensor signals. For example, the endoscope can include force sensors 228 and 229 configured to provide sensor signals representative of a measure force in each of the pull wires 202, 204, 206 and 208. The actuator controller can use the measure of force (e.g., as feedback) to control the respective motors 212 and 214.

In the example of FIGS. 18-21, the continuum mechanism 210 of the distal end portion 106 is configured to move along any of four orthogonal directions (e.g., up, down, left and/or right) based on the activation of the respective pull wires. A different number of pull wires can be used to increase or decrease the number of directions that the distal end portion can be moved. The amount of movement along each of the respective directions depends on the tension applied to (or not applied to) each of the pull wires, so that the deflection can be controlled over the spherical range of motion of the continuum mechanism 210. In an example, the continuum mechanism 210 is configured to provide the distal end portion 106 with approximately 270 degrees of deflection over a generally spherical range of motion.

In an example, each pair of pull wires is formed of a single length of a material that is flexible in a radial direction but substantially rigid in an axial direction. The pull wires 202, 204, 206 and 208 thus can be made of a non-stretching material, such as stainless steel, braided polymer fibers, or the like. The pull wires 202, 204, 206 and 208 can be carried in stainless steel or plastic sleeves or lumens within the proximal portion 108 so as to be protected from and to not interfere with other components that are muted through a central passage of the insertion part 102.

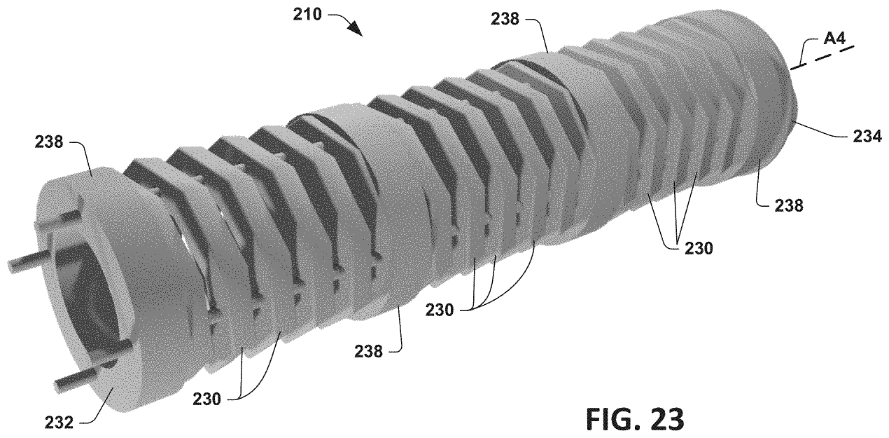

The continuum mechanism 210 includes a plurality of discs 230 axially spaced along the longitudinal axis A4 between respective proximal and distal ends 232 and 234 of the continuum mechanism 210. The continuum mechanism 210 can be implemented in a coreless configuration, such as shown in FIGS. 18-24, or as having a core. In the coreless configuration, the discs rest upon themselves without needing a spring core. In the example where the continuum mechanism has a core, the core can include a spring core surrounded by evenly spaced perforated rigid spacers (e.g., discs) through which respective tensile pull wires traverse such as diametrically opposed apertures formed through the disc.

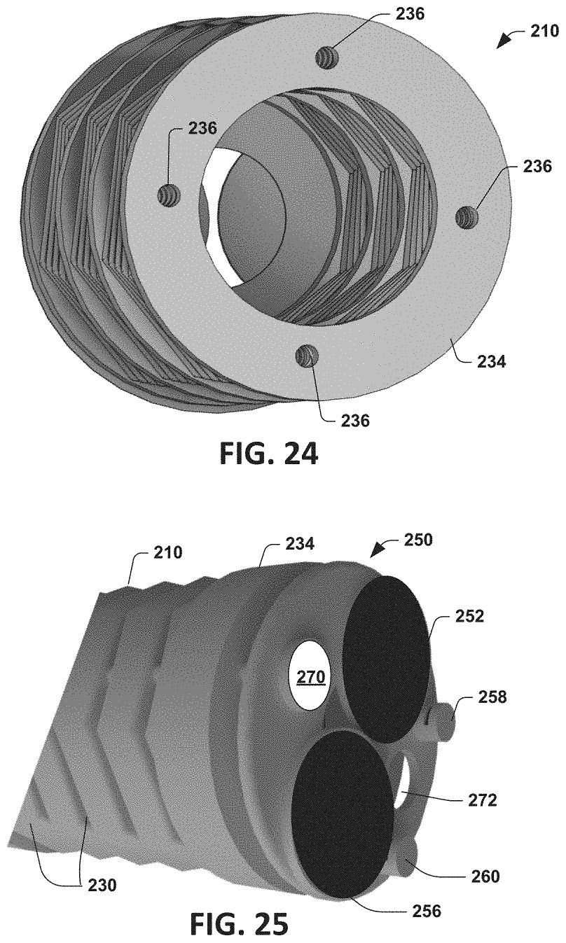

As best shown in FIG. 24, which shows the continuum mechanism 210 without pull wires, the continuum mechanism 210 includes guide holes 236 formed axially through the continuum mechanism. The guide holes 236 are configured for receiving a length of the respective pull wires 202. 204, 206 and 208. Each of pull wires 202, 204, 206 and 208 thus extends through a respective set of the axially aligned holes of the discs to hold the discs substantially orthogonally with respect to the longitudinal axis, including during steering of the distal end portion 106 based on actuating one or more of the pull wires. For example, the center of each hole 236 lies on the circumference of a virtual circle having a diameter that resides between radially inner and outer surfaces of the respective discs.

In some examples, the continuum mechanism 210 can include one or more annular supports 238, such as a number of such supports spaced axially apart along the length of the continuum mechanism. For example, annular supports 238 can be provided at the respective ends 232 and 234 as well as between the ends, such as placed at equally spaced apart axial positions. Each annular support 238 increases the stiffness of an adjacent part of the continuum mechanism 210, such as by connecting an adjacent pair (or more than two) of the discs together.

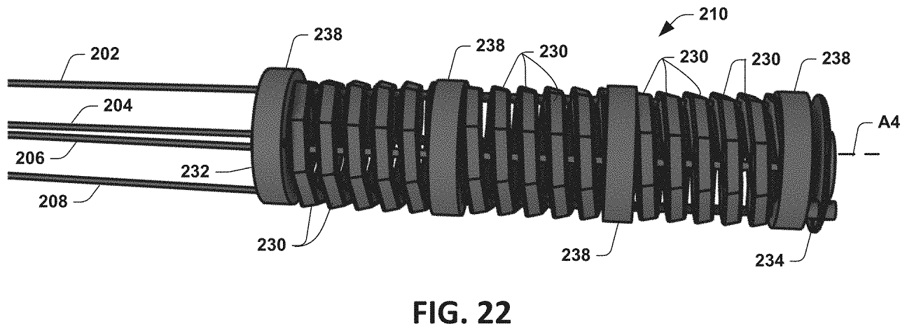

A distal end of each pull wire 202, 204, 206 and 208 can be anchored to the distal-most annular support 238 (or disc 230), such as by a connector (e.g., a weld, a fitting or other structure configured to anchor the distal end of the respective pull wires 202, 204, 206 and 208. The number of holes is at least equal to the number of pull wires. As shown in the example of FIGS. 22-24, there are four holes that can be arranged as hole-pairs in which each respective hole pair is diametrically opposed on the virtual circle. Other numbers of pull wires can be used in other examples.

In the example of FIGS. 22-24, the continuum mechanism 210 is formed of a helical sidewall in which each turn of the helical sidewall defines a respective disc 230 along its length. The pitch of the helical continuum mechanism 210 could be varied along its length to provide more (tighter pitch/shorter span between adjacent turns) or less (looser pitch/longer span between adjacent turns) flexibility to the continuum mechanism. For a given material composition having mechanical properties, the thickness (e.g., in its axial and/or radial direction) of the helical sidewall structure also can be configured to provide a desired flexibility and bendability relative to its central longitudinal axis.

As disclosed herein, the endoscope 100 can include one or more endoscopic tools configured to implement respective endoscopic functions. FIG. 25 shows an example of a tip 250 of the endoscope 100, which is coupled to the distal end of the continuum mechanism and adapted to perform various functions. The endoscope 100 can be equipped with any combination of tools to implement corresponding functions, such as including imaging, illumination, tip cleaning, gripping, cutting, ablation and suction.

For example, the endoscope 100 includes one or more imaging devices 252 and 256 located at the tip of the continuum mechanism. The imaging devices 252 and 256 can include any type of optical cameras such as digital video cameras, stereo cameras, high-speed cameras, light field cameras, and the like configured to provide image/video data to an image processor. Because the imaging devices 252 and 256 each capture image frames showing the position and/or orientation of the endoscope in at least two-dimensions, the two imaging devices can be configured and arranged to provide three-dimensional stereoscopic images as well as provide a depth measurement within the combined field of view. Each imaging device 252 and 256 can be implemented as a charge-coupled device (CCD) or complementary metal-oxide semiconductor (CMOS) camera. The imaging devices can include lenses configured to provide a wide focus and viewing angle. The lenses and/or an optically transparent window at the distal most end thereof can also be coated with anti-reflective material and a hydrophobic coating to reduce adhesion of blood and/or plasma to its exposed surface during use.

Additionally, the endoscope 100 can include one or more nozzles 258 and 260 adapted to supply a cleaning fluid onto the lens (or window) of a respective imaging device 252 and 256. A fluid channel (e.g., a conduit) can be coupled between each nozzle 258 and 260 and connector 264 (see, e.g., FIGS. 1 and 2). The connector 264 can be coupled to a. source of fluid (e.g., water or saline) that can be activated by a user input control (e.g., trigger 150) on the handle 112 to supply fluid for cleaning the lens of the camera at the tip of the endoscope while in use,

As also shown in FIG. 25, the endoscope 100 can include a light 270 configured to provide illumination for at least a portion of the field of view of the imaging devices. The light can be turned on to provide illumination continuously during use or it can be selectively activated, such as in response to activating a corresponding user input device. In one example, the light 270 includes one or more optical fibers extending through a passage within the endoscope insertion part 102 and coupled to light source, such as laser or light emitting diode (LED), such as can be located in a housing at the base of the robotic arm. In another example, the light 270 can be an LED that is electrically coupled (e.g., by an electrical cable) to a power source through a passage within the insertion part 102 to enable illumination to be provided from the tip 250.

The tip can also include one or more channels 272 such as providing a lumen or other type of passage. For example, the channel can be fluidly connected through a lumen with a proximal connector 262, which is configured to he coupled to a vacuum source or a fluid source. The source can be activated, such in response to activating a corresponding user input device, to provide flow of fluid (e.g., suction or irrigation through the channel). In another example, the channel can be used for passage of an instrument through the insertion part 102, such as a tool for performing a respective surgical function (e.g., cutting, ablating, electrical stimulation, biopsy, gripping, etc.)

A time of flight (TOF) sensor (e.g., LIDAR or TOF camera) can also be provided at the tip 250 of the endoscope 100, as well as at an anterior surface of the housing 110. The TOF sensors can be used to measure distance to a surface (e.g., tissue or other object).

In view of the foregoing, it is appreciated that the endoscopic devices, methods and systems can be used to address numerous issues experienced by surgeons and other endoscope users.

For example, surgeons hold the rigid endoscope for a prolonged period of time due to which they get fatigued. This results in pain and weakness in the arm of a surgeon. Some surgeons have voluntary and involuntary peripheral tremors which get transmitted to the endoscope when they hold it. This disturbs the field and creates a shaky view of the surgical field.

Certain anatomical structures are usually inaccessible to the rigid endoscope due to their anatomical structure and location. For example, after middle meatal antrostomy, the anterior wall of the maxillary sinus is extremely difficult to visualize for a zero degree endoscope. The surgeon has to use another endoscope with a 70 degree or a 90 degree viewing angle to visualize. Another example would be the base of the skull.

Ability to accurately locate a target anatomical structure is of paramount importance while dealing with certain anatomical structures, such as the internal carotid artery, optic nerve, tumors etc. Surgeons want to be notified (either with the help of an alarm/beeping sounds/lights or the like) when they are near an important anatomical structure. Correlation of a patient's anatomical structures per-operatively with their CT/MRI scan either in 2D or 3D view in a monitor or 3D augmented reality (AR)/virtual reality (VR) eye glasses.

The endoscope also can facilitate hands free surgery. In an example emergency scenario, surgeons usually hold the rigid endoscope in their left hand and a surgical instrument in the right hand. Sometimes both hands are needed to either operate or control bleeding in the surgical site. In such a scenario an assistant is needed to hold the endoscope in the right orientation and location. In an example regular surgery scenario, the surgery can be hands free in the sense either hands are not being used to hold the endoscope. This is a value addition in certain surgeries which can benefit greatly if the surgeon uses two hands to operate instead of one. One example would be to raise endoscopic endonasal flaps.

The endoscope also exhibits improved ergonomics as it is equally useful for holding by the right or left hand. A left handed person holding an endoscope is different from a right handed person doing the same. This becomes important when the assistant is either left handed or right handed. Depending on that they will have to change their relative position in the operation theatre with respect to the operating table.

When operating near important structures, depth is important. For example, debriders are instruments with a rotating tip which are adapted to cut the tissue, and can also suck out cut or other loose tissue. The information on how far a surgeon should advance or retract the tip can be aided by using depth information, such as real-time 3D visualization instead of 2D visualization.

Robot assisted surgeries have traditionally been console-based, where the surgeon sits in a console far away from a patient and the robotic arms with various specialized features are positioned inside the patient. The surgeon controls the robotic arm with the help of finger controls and foot pedal in the console. This is good for abdominal procedures. But when it comes to ENT, head and Neck, Neurosurgery, Ophthalmology procedures having a robot assisted surgical system that can have features of both traditional hand-held manual endoscopic surgery and also a robot assisted one for a better surgical outcome of the patient. The endoscope 100 enables a hybrid approach in which the surgery can be performed manually and/or robotically assisted.

Certain diseases of the brain demand extreme high levels of precision. For example, a tumor present in the brain tissue is often surrounded by normal neurons. The challenge here is to first identify the location, size and relative position of the tumor and second would be to chart a path of least injury to the surrounding neurons. 3D stereoscopic imaging provided by the endoscope can facilitate these and other scenarios.

The rigid endoscope's tip is often occluded by blood and other fluids and tissues. While doing a surgical procedure, if blood gets accumulated onto the tip of the endoscope, the surgeon has to stop the procedure, and then remove the endoscope outside the patient's nose, clean the tip and then re-insert it in the nose and continue the procedure. This process of manual cleaning happens repeatedly 50 to 60 times during a single procedure and takes a significant time of the surgery. Automating this task, such as disclosed herein, can reduce the surgery time significantly.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques and controls may be implemented in hardware, software, firmware, or any combination thereof If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

As used herein, phrases and/or drawing labels such as “X-Y”, “between X and Y” and “between about X and Y” can be interpreted to include X and Y.

As used herein, phrases and/or drawing labels such as “between about X and Y” can mean “between about X and about Y.”

As used herein, phrases and/or drawing labels such as “from about X to Y” can mean “from about X to about Y.”

In this description and claims when an element is referred to as being “on,” “attached” to, “connected” to, “coupled” with, “contacting”, “adjacent”, etc., another element, it can be directly on, attached to, connected to, coupled with, contacting, or adjacent the other element or intervening elements may also be present. In contrast, when an element is referred to as being, for example, “directly on,” “directly attached” to, “directly connected” to, “directly coupled” with, “directly contacting”, or “directly adjacent” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

The recitation “based on” means “based at least in part on.” Therefore, if X is based on Y, X may be a function of Y only or a function of Y and any number of other factors.

Modifications are possible in the described embodiments, and other embodiments are possible, within the scope of the claims.

1. An endoscope, comprising:
an elongated handle extending between first and second ends;
an elongated insertion part coupled to and extending from a proximal end portion to terminate in a distal end portion;
a housing coupled to the first end of the handle, the housing including an actuator therein configured to cause movement of the distal end portion of the insertion part; and
a mount coupling on the housing or the handle, the mount coupling adapted to connect to a mating part of a support structure, such that when connected to the support structure, the endoscope is held in a fixed position with respect to the mating part of the support structure, and when not connected to the support structure, the endoscope can be manipulated manually independently from the support structure.

2. The endoscope of claim 1, wherein at least one of the mount coupling and the mating part comprises a fastener adapted to releasably lock the mount coupling with respect to the support structure and to enable release of the endoscope from the support structure in response to actuation of the fastener.

3. The endoscope of claim 1, wherein:
the mount coupling comprises a magnet; and/or
the mount coupling is located on a surface of the housing.

4. The endoscope of claim 1, further comprising:
an adjustment coupling between the housing and the handle, the adjustment coupling configured to enable angular movement of the handle with respect to the housing and/or the insertion part.

5. The endoscope of claim 4, further comprising a lock configured to fix a relative position between the handle and the housing and/or the insertion part.

6. The endoscope of claim 1, further comprising:
a steering interface device mounted on the handle, the steering interface device configured to provide a steering signal based on an applied user input; and
an actuator controller configured to control the actuator responsive to the steering signal to move the distal end portion in a respective direction relative to a central axis of the insertion part.

7. The endoscope of claim 6, wherein the steering interface device comprises at least one of:
a joystick, in which the joystick is arranged to extend from a side of the handle in a direction that is transverse to a longitudinal axis of the handle;
a trigger configured to provide an activation signal to activate a respective endoscopic function; and
an arrangement of push buttons, in which each of the push buttons is adapted to activate a respective switch, and each switch is configured to provide a respective steering signal to the actuator controller based on which the actuator controller controls the actuator.

8. The endoscope of claim 1, wherein the distal end portion of the elongated insertion part comprises a continuum mechanism extending longitudinally between proximal and distal ends thereof, and the continuum mechanism is adapted to enable the distal end thereof to deflect over a generally spherical range of motion.

9. The endoscope of claim 8, wherein the continuum mechanism comprises:
a plurality of discs axially spaced along a longitudinal axis between respective proximal and distal ends thereof; and
a tubular outer sheath of a flexible material disposed over an outer surface of the discs.

10. The endoscope of claim 9, wherein the plurality of discs between the proximal and distal ends thereof have respective holes extending axially through the discs, a respective hole of each of the discs between the proximal and distal ends being aligned axially to provide a set of aligned holes, the endoscope further comprising:
an arrangement of pull wires, each pull wire extending through a respective set of axially aligned holes of the discs to hold the discs substantially orthogonally with respect to the longitudinal axis during steering of the distal end portion based on actuating one or more of the pull wires.

11. The endoscope of claim 10, wherein the actuator comprises a rotary motor having a motor shaft configured to rotate responsive to a motor control signal, the endoscope further comprising:
an actuator controller configured to provide the motor control signal responsive to a steering command signal; and
a pulley member adapted to rotate about an axis based on rotation of the motor shaft, a proximal end of the pull wire coupled to the pulley member so that rotation of the pulley member causes corresponding deflection of the distal end portion.

12. The endoscope of claim 1, further comprising a gimbal apparatus coupled between the housing and the handle, the gimbal apparatus configured to stabilize a position and orientation of the insertion part of the endoscope.

13. The endoscope of claim 12, wherein the gimbal apparatus is a motorized gimbal apparatus comprising:
an inertial measurement unit in the housing, the inertial measurement unit configured to provide inertial sensor data representative of position and/or motion of the housing;
a plurality of motors, each configured to rotate the housing about a respective axis of rotation, in which the respective axes of rotation are orthogonal to each other; and
a gimbal controller configured to provide motor control signals to control the plurality of motors for stabilizing the insertion part based on the inertial sensor data.

14. A robotic endoscopic system comprising:
the endoscope of claim 1; and
a robotic arm that constitutes the support structure, in which the mating part is coupled to the robotic arm to hold the endoscope at a fixed position with respect to the robotic arm.

15. An robotic endoscope system, comprising a robotic arm including a mating coupling part on the robotic arm; and
an endoscope comprising:
an elongated handle extending between first and second ends;
an elongated insertion part coupled to and extending from a proximal end portion to terminate in a distal end portion;
a housing coupled to the first end of the handle, the housing including an actuator therein configured to cause movement of the distal end portion of the insertion part; and
a mount coupling on the housing or the handle, the mount coupling adapted to connect to the mating coupling part of the robotic arm, such that when connected to the robotic arm, the endoscope is held in a fixed position with respect to the robotic arm, and when removed from the robotic arm, the endoscope can be manipulated manually independently from the robotic arm.

16. The robotic endoscope system of claim 15, wherein at least one of the mount coupling and mating part comprises a fastener adapted to releasably lock the mount coupling with respect to the mating part and to enable release of the endoscope from the robotic arm in response to actuation of the fastener.

17. A method, comprising
disconnecting an endoscope from an attachment to a robotic arm, in which the attachment to the robotic arm includes a mount coupling, which is on a housing or handle of the endoscope and releasably coupled to a mating part of the robotic arm to fix the endoscope with respect to the robotic arm;
manually manipulating the endoscope independently from the robotic arm, in which the endoscope includes an elongated insertion part having a distal end portion, and the endoscope includes an actuator configured to move the distal end portion of the elongated insertion part in response to a steering control signal; and
re-connecting the endoscope to the robotic arm by connecting the mount coupling to the mating part of the robotic arm, such that when connected to the robotic arm, the endoscope is held in a fixed position with respect to the mating part of the robotic arm and moveable commensurate with movement of the robotic arm.

18. The method of claim 17, wherein the handle is coupled to the housing through an adjustment coupling to enable rotation of the handle relative to the housing and/or the insertion part, and the method further comprises:
rotating the handle about the adjustment coupling relative to the housing and/or the insertion part, and
fixing a relative position between the handle and the housing and/or the insertion part.

19. The method of claim 17, further comprising:
providing the steering control signal based on user input applied at a steering user interface device on the handle; and
controlling, by an actuator controller, the actuator responsive to the steering control signal to move the distal end portion in a respective direction relative to a central axis of the insertion part.

20. An endoscope, comprising:
an elongated handle extending between first and second ends;
an elongated insertion part coupled to and extending from a proximal end portion to terminate in a distal end portion;
a housing coupled to the first end of the handle, the housing including an actuator therein configured to cause movement of the distal end portion of the insertion part, wherein the distal end portion comprises a continuum mechanism extending longitudinally between proximal and distal ends thereof, the continuum mechanism configured to enable the distal end thereof to deflect over a generally spherical range of motion;
a mount coupling on the housing or the handle, the mount coupling adapted to connect to a mating part of a support structure, such that when connected to the support structure, the endoscope is held in a fixed position with respect to the mating part of the support structure, and when not connected to the support structure, the endoscope can be manipulated manually independently from the support structure;
a steering interface device mounted on the handle, the steering interface device configured to provide a steering signal based on an applied user input; and
an actuator controller configured to control the actuator responsive to the steering signal to move the distal end portion in a respective direction relative to a central axis of the insertion part; and
at least one endoscopic tool at the distal end of the endoscope.
